(19) United States
(12) Patent Application Publication
(10) Pub. No.: US 2024/0366827 A1
(43) Pub. Date: Nov. 7, 2024

(54) COMPOSITION FOR WET INDICATOR AND WET INDICATOR

(71) Applicant: Henkel AG & CO. KGaA, Duesseldorf (DE)

(72) Inventor: Shigekazu Saito, Osaka (JP)

(21) Appl. No.: 18/735,400

(22) Filed: Jun. 6, 2024

Related U.S. Application Data
(63) Continuation of application No. PCT/IB2023/051063, filed on Feb. 7, 2023.

(30) Foreign Application Priority Data
Dec. 10, 2021 (JP) 2021-201008

(51) Int. Cl. A61L 15/22; A61L 15/56; A61L 15/60; C09B 9/00

(52) U.S. Cl. CPC A61L 15/225; A61L 15/56; A61L 15/60; C09B 9/00

(57) ABSTRACT

Problem to be solved: To provide a composition for wet indicator excellent in discoloration performance and compatibility, and having suppressed odor.

Means for solution: A composition for wet indicator comprising an oily gel containing a liquid oily substance and a gelling agent, a colorant and a water holding agent.

### CROSS-REFERENCE TO RELATED APPLICATION

This application claims benefit under Article 4 of the Paris Convention based on Japanese Patent Application No. 2021-201008 filed in Japan on Dec. 10, 2021, incorporated herein by reference in its entirely.

### TECHNICAL FIELD

The present invention relates to a wet indicator which is a member that notifies the presence of moisture by discoloration, and a composition used as a member of the wet indicator.

### BACKGROUND ART

Examples of an article that exhibits a function by being in contact with moisture include a moisture exchanger (HEM), a moisture detector, an autoclave (sterilization) tape, a packaging article and an absorbent article. As a device that displays that these articles are in a wet state and exhibit a function, a wet indicator is known. Wetting refers to a state in which a liquid comprising water such as a body fluid, that is, moisture is in contact. The indicator refers to a display tool and a marking tool.

Generally, the wet indicator changes its color to indicate whether the article is dry or wet with moisture. For example, the wet indicator is colored or discolored when the article is wet, thereby displaying a status of wet state of the article.

Patent Documents 1 to 3 describe a composition for wet indicator used in combination with an absorbent article such as a diaper.
Patent Document 1 describes a wet indicator composition that changes color in response to pH change. The wet indicator composition contains a water-insoluble thermoplastic polymer composition, a superabsorbent polymer, a wet indicator and a surfactant. ([Abstract], [Claim 1], [Claim 2]).
Patent Document 2 describes a hot melt damp indicator composition that may be applied using a conventional hot melt applicator device. This hot melt damp indicator composition contains components such as a water-insoluble thermoplastic polymer, an anionic surfactant and a leuco dye ([Abstract], [Claim 1], [0006]).
Patent Document 3 describes a dampness/fluid indicator composition that is colorless in its initial state and may provide various final wet color options in the presence of water. The dampness/fluid indicator composition includes a leuco dye and a color-developing agent in a hot melt adhesive matrix ([Abstract], [0001], [0008]).

### PRIOR ART DOCUMENTS

### Patent Documents

Patent Document 1: JP 2009-511673 A
Patent Document 2: JP 2018-515165 A
Patent Document 3: JP 2018-517894 A

### SUMMARY OF THE INVENTION

### Problems to be Solved by the Invention

However, the compositions for indicators in Documents 1 to 3 are mainly composed of thermoplastic resins such as styrene block polymers, which do not have sufficiently excellent water permeability. If the compositions for wet indicator have poor water permeability, the degree of discoloration is weak, and timing of discoloration may also be delayed when they are brought into contact with moisture.

Furthermore, the compositions for indicator of Documents 1 to 3 contain plasticizers (tackifiers) such as rosin to increase the compatibility of blending components. However, many of the tackifiers such as rosin that improve compatibility have a strong odor and may give a feeling of discomfort to the user.

The present invention solves such problems, and an object thereof is to provide a composition for wet indicator which is excellent in discoloration performance and compatibility, and is also suppressed in odor.

As a result of intensive study, the present inventors found that a composition comprising an oily gel, a colorant and a water holding agent improves the discoloration performance of the wet indicator, has excellent compatibility of each component, and suppresses odor.

### SUMMARY OF THE INVENTION

That is, the present invention, and preferred aspects of the present invention are as follows.
1. A composition for wet indicator comprising an oily gel containing a liquid oily substance and a gelling agent, a colorant and a water holding agent.
2. The composition for wet indicator according to the above 1, wherein the water holding agent comprises a higher alcohol derivative.
3. The composition for wet indicator according to the above 2, wherein the higher alcohol derivative comprises at least one selected from stearyl alcohol, rice wax and carnauba wax.
4. The composition for wet indicator according to any one of the above 1 to 3, wherein the oily substance is at least one selected from the group consisting of paraffin oil, naphthene oil and aromatic oils.
5. The composition for wet indicator according to any one of the above 1 to 4, wherein the gelling agent comprises at least one selected from the group consisting of saturated fatty acids and saturated fatty acid derivatives, which have 16 or more carbon atoms.
6. The composition for wet indicator according to any one of the above 1 to 5, wherein the colorant comprises a leuco dye or a pH indicator.
7. The composition for wet indicator according to any one of the above 1 to 6, further comprising a surfactant.
8. The composition for wet indicator according to any one of the above 1 to 7, further comprising a tackifier resin.
9. A wet indicator comprising the composition for wet indicator according to any one of the above 1 to 8.
10. An absorbent article comprising the wet indicator according to the above 9.

### Effects of the Invention

The composition for wet indicator of the present invention is excellent in discoloration performance and compatibility, and is suppressed in odor. The wet indicator comprising the composition for wet indicator of the present invention is suitable as a member of an absorbent article such as a diaper.

### DESCRIPTION OF EMBODIMENTS

The composition for wet indicator of the present invention comprises an oily gel, a colorant and a water holding agent.

### <Oily Gel>

The composition for wet indicator of the present invention comprises an oily gel. The oily gel includes a liquid oily substance and a gelling agent. It can be said that the oily gel is a swollen body having a structural body of a three-dimensionally crosslinked gelling agent and a liquid oily substance confined in the structural body.

Because the composition for wet indicator of the present invention contains an oily gel, the composition for wet indicator has appropriate hardness that is softer than that of a hot-melt type composition for wet indicator, and moisture so easily penetrates into the composition, that the composition is strongly and quickly discolored. Thereby, with the wet indicator of the present invention, a status of wet state of the article may be accurately displayed. In addition, because the oily gel is able to confine a colorant or a body fluid that causes odor, it is possible to reduce the odor of the absorbent article of the present invention.

The liquid oily substance refers to a lipophilic substance that is liquid at room temperature, is incompatible with water, and is compatible with a nonpolar solvent. For example, wax is an oily substance but is solid at room temperature and thus does not correspond to a liquid oily substance. Since a solid oily substance is poor in water permeability, when used in a matrix of the composition for wet indicator, the solid oily substance is likely to adversely affect discoloration properties of the wet indicator. Examples of the liquid oily substance generally include hydrocarbon oils, liquid polymers and the like.

The hydrocarbon oils include paraffin oil, naphthene oil and aromatic oils. Weight average molecular weight of the hydrocarbon oil is preferably from 200 to 2000, from the viewpoint of improving water permeability. The hydrocarbon oil preferably contains at least one selected from paraffin oil and naphthene oil, and more preferably contains paraffin oil, from the viewpoint of improving compatibility with the colorant.

As the hydrocarbon oil, a commercially available product may be used. Examples of the commercially available hydrocarbon oil include White Oil Broom 350 (trade name) manufactured by Kukdong Oil & Chemicals Co. Ltd., Diana Fresia PW32 (trade name), Diana Process Oil PW-90 (trade name) and Daphne Oil KP-68 (trade name) manufactured by Idemitsu Kosan Co., Ltd., Nyflex 222B (trade name) manufactured by Nynas AB, SUNPURE N90 manufactured by JAPAN SUN OIL COMPANY, LTD., KN4010 (trade name) manufactured by PetroChina Company Limited, Enerper M1930 (trade name) manufactured by BP Chemicals Ltd., Kaydol (trade name) manufactured by Crompton Corporation and Primol 352 (trade name) manufactured by Esso Standard Oil Co.

The liquid polymer refers to a polymer that exhibits a liquid state at room temperature. The type of the polymer is not particularly limited, but is generally at least one polymer selected from polybutene, polybutadiene, polyisobutylene and polyisoprene. The liquid polymer preferably has a weight average molecular weight of from 2000 to 100000 and more preferably from 2000 to 80000, from the viewpoint of improving water permeability. The liquid polymer preferably contains polybutene, from the viewpoint of improving compatibility with the colorant.

As the liquid polymer, a commercially available product may be used. Examples of the liquid polymer which is a commercially available product include Nisseki Polybutene HV-300 (trade name) and Nisseki Polybutene HV-1900 (trade name) manufactured by ENEOS Corporation, B-1000 (trade name) and BI-2000 (trade name) manufactured by Nippon Soda Co., Ltd., Tetrax (trade name) and Himol (trade name) manufactured by JX Energy Corporation, LIR-15 (trade name) and LIR-50 (trade name) manufactured by KURARAY CO., LTD. and Nipol IR2200 (trade name) manufactured by Zeon Corporation.

The liquid oily substance is contained in the composition for wet indicator in an amount of preferably from 10 to 95% by mass, more preferably from 15 to 90% by mass, and still more preferably from 17 to 85% by mass, based on the total amount of the oily gel, the colorant and the water holding agent. By adjusting the content of the liquid oily substance in the composition for wet indicator within the above ranges, compatibility between the liquid oily substance and the colorant is improved, moisture is easily brought into contact with the colorant, and the composition for wet indicator of the present invention may exhibit clear and rapid coloration.

The liquid oily substance is confined in the oily gel by being held by the three-dimensionally crosslinked gelling agent. The gelling agent that is three-dimensionally crosslinked in the presence of the liquid oily substance may be a known substance. The gelling agent is a chemical substance that converts liquid into gel. The gelling agent is preferably a saturated fatty acid having 16 or more carbon atoms and derivatives of the saturated fatty acid, from the viewpoint of improving water permeability.

The saturated fatty acid refers to a fatty acid not having double bond or triple bond in the carbon chain. The fatty acid is an aliphatic carboxylic acid having at least one carboxyl group. A saturated fatty acid having a hydroxyl group is preferable because it is easily crosslinked, and has excellent performance of converting the entire composition for indicator into gel. The saturated fatty acid is preferably in the form of a chain. Moreover, the chain form saturated fatty acid is preferably a linear saturated fatty acid.

The saturated fatty acid has more preferably from 16 to 36 carbon atoms, and still more preferably from 18 to 20 carbon atoms. Specific examples of the saturated fatty acid having 16 or more carbon atoms include arachidic acid having 20 carbon atoms, stearic acid having 18 carbon atoms, 12-hydroxystearic acid having 18 carbon atoms, margaric acid having 17 carbon atoms, palmitic acid having 16 carbon atoms and 16-hydroxyhexadecanoic acid having 16 carbon atoms. Among them, 12-hydroxystearic acid is particularly preferable.

The saturated fatty acid derivative refers to a compound in which a part of the saturated fatty acid is substituted with a group which can coexist. The saturated fatty acid derivative may have 16 or more carbon atoms, preferably from 16 to 36 carbon atoms, and more preferably from 18 to 20 carbon atoms. For example, a fatty acid amide, fatty acid alkyl ester, fatty acid metal salt, monoglyceride, diglyceride, triglyceride, sorbitan fatty acid ester or diglycerin fatty acid ester having 16 or more carbon atoms may be used as the saturated fatty acid derivative. Preferred saturated fatty acid derivatives include saturated fatty acid metal salts.

The saturated fatty acid derivative particularly preferably has a chemical structure derived from stearic acid. The “chemical structure derived from stearic acid” refers to the unit represented by formula

CH₃ (CH₂)₁₆COOH (1). The saturated fatty acid derivative also includes a chemical structure in which a part of formula (1) is substituted with another group which can coexist (for example, a hydroxyl group, an alkyl group, an alkali metal, or an alkaline earth metal) and an oligomer or a polymer having the unit represented by formula (1).

The fatty acid metal salt is preferably a metal salt having a chemical structure derived from stearic acid, and specific examples thereof include sodium stearate and lithium 12-hydroxystearate having 18 carbon atoms, and magnesium stearate having 36 carbon atoms. Among them, lithium 12-hydroxystearate is particularly preferable.

Glycerides include castor oil and castor hardened oil. Castor oil is a glyceride of unsaturated fatty acids (recinoleic acid, oleic acid, linoleic acid) and saturated fatty acids (palmitic acid, stearic acid, 12-hydroxystearic acid). Castor hardened oil is a hydrogenated product of castor oil.

When the composition for wet indicator of the present invention contains 12-hydroxystearic acid and lithium 12-hydroxystearate as a gelling agent, it becomes easy to solidify the oily substance to prepare an oily gel having appropriate hardness. When the oily gel is formed to have appropriate hardness, moisture easily penetrates into the gel. Because the moisture easily permeates, the moisture and the surfactant easily coexist, generation of protons is facilitated, and discoloration of the colorant is promoted.

The gelling agent is blended with the oily substance to form a continuous porous body like tissue, and forms a structure in which the oily substance is closed in voids of the continuous porous body. By blending the liquid oily substance with the gelling agent, an oily gel having appropriate hardness is obtained, and the oily gel has a fine continuous porous body. In the composition for wet indicator of the present invention, since the oily gel has a fine continuous porous structure, for example, it is possible to easily permeate moistures such as urine and body fluid.

The gelling agent is contained in the composition for wet indicator in an amount of preferably from 2 to 80% by mass, more preferably from 5 to 70% by mass, and still more preferably from 10 to 50% by mass, based on the total amount of the oily gel, the colorant and the water holding agent. By adjusting the content of the gelling agent in the composition for wet indicator within the above ranges, compatibility between the liquid oily substance and the colorant is improved, moisture and the colorant are easily brought into contact with each other, and the composition for wet indicator of the present invention may exhibit clear and rapid coloration.

The composition for wet indicator of the present invention contains the oily gel in an amount of preferably from 50 to 98 parts by mass, more preferably 59 to 95 parts by mass, and still more preferably 65 to 90 parts by mass, based on 100 parts by mass of the total amount of the oily gel, the colorant, and the water holding agent. By adjusting the content of the oily gel within the above ranges, the composition for wet indicator of the present invention can make it difficult for body fluids and colorants to be eluted to the outside.

Parts by mass of the oily gel is the sum of parts by mass of the gelling agent and parts by mass of the oily substance.

In the present specification, the colorant is defined as a substance that colors or changes color due to changes in hydrogen ion concentration, such as proton sensitivity.

The colorant includes a dye, an indicator and the like. Specific examples of the colorant that may be used include oxazolidine-based dyes, azo-based dyes, methine-based dyes, anthraquinone-based dyes, leuco dyes and the like. The colorant preferably contains a leuco dye and a pH indicator, from the viewpoint of obtaining rapid and clear discoloration. Since the leuco dye or the pH indicator has strong proton sensitivity, use of the leuco dye or the pH indicator improves discoloration performance of the composition for wet indicator.

The leuco dye is a dye that may change between two chemical species (one of which is colorless). Reversible transformations are caused by heat, light or pH; resulting in examples of thermochromism, photochromism and halochromism, respectively. Typical irreversible transformations are caused by reduction or oxidation. The colorless form is sometimes referred to as a leuco form.

The leuco dye is not limited as long as it is sufficiently colored, and a known or commercially available one may be used. For example, the following compounds such as a leuco dye that may develop color by an acid may be suitably used. These may be used singly or two or more kinds thereof.
(a) Fluorans: 2′-[(2-chlorophenyl)amino]-6′-(dibutylamino)-spiro [isobenzofuran-1 (3H),9′-(9H) xanthen]-3-one, 3-diethylamino-6-methyl-7-chlorofluoran, 3-dimethylaminobenzo (a)-fluoran, 3-amino-5-methylfluoran, 2-methyl-3-amino-6,7-dimethylfluoran, 2-bromo-6-cyclohexylaminofluoran, 6′-ethyl (4-methylphenyl)amino-2′-(N-methylphenylamino)-spiro (isobenzofuran1 (3H),9′-(9H) xanthen)-3-one, 3,6-diphenylaminofluoran, 9-ethyl (3-methylbutyl)amino-spiro [12H-benzo (a) xanthene-12,1′ (3′H) isobenzofuran]-3′-one, 2′-[bis(phenylmethyl)amino]-6′-(diethylamino)-spiro-[isobenzofuran-1 (3H),9′-(9H) xanthen]-3-one and the like;
(b) Fluorenes: 3,6-bis(diethylamino) fluorenespiro(9,3′)-4′-azaphthalide, 3,6-bis(diethylamino) fluorenespiro(9,3′)-4′,7′-diazaphthalide and the like;
(c) Diphenylmethane phthalides: 3,3-bis-(p-ethoxy-4-dimethylaminophenyl) phthalide and the like;
(d) Diphenylmethane azaphthalides: 3,3-bis-(1-ethoxy-4-diethylaminophenyl)-4-azaphthalide, 3,3-bis(4-diethylamino-2-ethoxyphenyl)-4-azaphthalide and the like;
(e) Indolyl phthalides: 3,3-bis(n-butyl-2-methylindol-3-yl) phthalide, 3,3-bis(1-ethyl-2-methylindol-3-yl) phthalide and the like;
(f) Phenylindolyl phthalides: 3-(1-diethylaminophenyl)-3-(1-ethyl-2-methylindol-3-yl) phthalide and the like;
(g) Phenylindolyl azaphthalides: 3-(2-ethoxy-4-diethylaminophenyl)-3-(1-ethyl-2-methylindol-3-yl)-4-azaphthalide, 3-[2-ethoxy-4-(N-ethylanilino)phenyl]-3-(1-ethyl-2-methylindol-3-yl)-4-azaphthalide and the like;
(h) Styrylquinolines: 2-(3-methoxy-4-dodecoxystyryl) quinolone and the like;
(i) Diazarhodamine lactones: 2-(dimethylamino)-8-(dimethylamino)-4-methyl-spiro [5H-(1)benzopyrano (2,3-d)pyrimidine-5,1′ (3′H)-isobenzofuran] and the like;
(j) Pyridines: 2,6-diphenyl-4-(6-dimethylaminophenyl)pyridine, 2,6-diethoxy-4-(4-diethylaminophenyl)pyridine and the like;
(k) Quinazolines: 2-(4-N-methylanilinophenyl)-1-phenoxyquinazoline, 2-(4-dimethylaminophenyl)-4-(1-methoxyphenyloxy) quinazoline and the like;
(l) Bisquinazolines: 4,4′-(ethylenedioxy)-bis [2-(1-diethylaminophenyl) quinazoline], 4,4′-(ethylenedioxy)-bis [2-(1-di-n-butylaminophenyl) quinazoline] and the like;
(m)Ethylenophthalides: 3,3-bis [1,1-bis-(p-dimethylaminophenyl)ethyleno-3]phthalide and the like;
(n) Ethylenoazaphthalides: 3,3-bis [1,1-bis-(p-dimethylaminophenyl)ethyleno-2]-4-azaphthalide, 3,3-bis [1,1-bis-(p-dimethylaminophenyl)ethyleno-2]-4,7-diazaphthalide and the like; and
(o) Aminophthalides: 3,3-bis(p-dimethylaminophenyl)-6-dimethylaminophthalide (crystal violet lactone) and the like.

In addition, at least one of aminophthalides and fluorans may be used as a colorant. As the fluoran, particularly, 2′-[bis(phenylmethyl)amino]-6′-(diethylamino)-spiro-[isobenzofuran-1 (3H),9′-(9H) xanthen]-3-one, 2′-[(2-chlorophenyl)amino]-6′-(dibutylamino)-spiro [isobenzofuran-1 (3H),9′-(9H) xanthen]-3-one and 3,6-diphenylaminofluoran are more preferable. As the aminophthalides, crystal violet lactone is particularly preferable.

When the composition for wet indicator of the present invention contains a leuco dye as a colorant, the leuco dye is easily ring-opened by protons generated in the presence of a surfactant and urine (moisture), and the composition for wet indicator is quickly colored, which is preferable. The content of the leuco dye may be appropriately determined according to the type of leuco dye, desired hue and the like.

The pH indicator refers to an agent to be added to a reaction liquid in order to know an equivalent point by a change directly visible, such as a change in color or formation of a precipitate in titration. As the pH indicator, an acid-base indicator is used. Specific examples of the pH indicator include phenolphthalein, methyl orange, potassium chromate, bromocresol green, bromophenol blue and the like.

The colorant is contained in the composition for wet indicator in an amount of preferably from 0.01 to 7% by mass, more preferably from 0.05 to 5% by mass, and still more preferably from 0.1 to 4% by mass, based on the total amount of the oily gel, the colorant and the water holding agent. By adjusting the content of the colorant in the composition for wet indicator within the above ranges, color difference between the dry state and the wet state of the composition for wet indicator increases, and discoloration may be easily perceived.

The colorant is used in combination with a surfactant described below to have a more excellent discoloration effect. The surfactant improves compatibility of the colorant with other components, and the colorant is easily confined in the oily gel.

### <Water Holding Agent>

The “water holding agent” is a substance that has the function of holding water. In general, the water holding agent is a compound having a chemical structure derived from a hydroxyl group. Examples of the water holding agent include higher alcohol derivatives.

The composition for wet indicator of the present invention contains a water holding agent, and thereby the compatibility of each component is improved. In addition, the holding of moisture in the composition for wet indicator facilitates contact of moisture with the colorant, so that the discoloration of the colorant is promoted.

The “higher alcohol” is usually an aliphatic alcohol having 6 or more carbon atoms. In the present specification, the “higher alcohol derivative” is a generic term for compounds having a chemical structure derived from a higher alcohol, and indicates pure higher alcohols and compounds obtained by partially changing pure higher alcohols.

The “higher alcohol derivative” includes, specifically, higher alcohols, esters of higher alcohols and fatty acids, polymers based on higher alcohols, and modified products in which a part of the chemical structure of a higher alcohol is substituted with another functional group.

The “hydroxyl group” refers to a functional group having the structure represented by the formula-OH. The “chemical structure derived from a hydroxyl group” is a generic term for a chemical structure in which a part of the hydroxyl group, for example a hydrogen atom, is substituted with another functional group. A specific example of the “chemical structure derived from a hydroxyl group” includes an ester bond (ester group) produced by the condensation reaction of an acid and an alcohol.

In the present invention, the water holding agent preferably has an aliphatic alcohol derivative having 10 or more carbon atoms, particularly preferably an aliphatic alcohol derivative having from 12 to 35 carbon atoms, and most preferably an aliphatic alcohol derivative having from 16 to 34 carbon atoms.

The “aliphatic alcohol” is a generic term for alcohols whose basic structure is a linear hydrocarbon, usually having 8 or more carbon atoms.

The “aliphatic alcohol derivative” is a generic term for a compound having a chemical structure derived from the aliphatic alcohol, and specifically includes aliphatic alcohols, esters of aliphatic alcohols and fatty acids, polymers based on aliphatic alcohols, modified products in which a part of the chemical structure of the aliphatic alcohol is substituted with another functional group and the like.

Specific examples of the water holding agent include:
aliphatic alcohols such as capryl alcohol (1-octanol), capric alcohol (1-decanol), lauryl alcohol, myristyl alcohol, tridecyl alcohol, cetanol, stearyl alcohol, ceryl alcohol (1-hexacosanol), myricyl alcohol (1-triacontanol), and gedyl alcohol (1-tetraacontanol);
esters of aliphatic alcohols and fatty acids such as rice wax, carnauba wax, and candelilla wax; and the like.

The main components of rice wax are esters of fatty acids and higher alcohols. The fatty acids consist of palmitic acid (C16), behenic acid (C22), lignoceric acid (C24) and other fatty acids. The higher alcohols consist mainly of 1-hexacosanol (C26) and triacontanol (C30).

Carnauba wax is composed of aliphatic esters (40% by mass), diesters of 4-hydroxycinnamic acid (21.0% by mass), ω-hydroxy carboxylic acid (13.0% by mass), aliphatic alcohols (12% by mass), and the like. These compounds are mainly derivatives of acids and alcohols having carbon atoms ranging from 26 to 30.

In the present invention, the water holding agent preferably contains at least one selected from stearyl alcohol, rice wax, and carnauba wax. When the water holding agent contains the substances mentioned above, the composition for wetting indicator may improve the discoloration performance of the wetting indicator at a higher level, have excellent compatibility of the respective components, and reduce odor.

The water holding agent is included in the composition for wet indicator in an amount of preferably from 2 to 50 parts by mass, more preferably from 5 to 40 parts by mass, and still more preferably from 10 to 35 parts by mass, based on the total amount of the oil gel, the colorant and the water retention agent. By adjusting the content of the gelling agent in the composition for wet indicator within the above ranges, the water and the colorant are so easily brought into contact with each other, that the composition for wet indicator of the present invention may exhibit clear and rapid coloration.

### <Surfactant>

Surfactants are substances that increase the contact between the oily substance and water in the presence of moisture to increase the degree of discoloration of colorants. The greater degree of discoloration of the colorant allows the composition for wet indicator to clearly indicate the wetting state.

For example, anionic surfactants and nonionic surfactants are preferable because they are easily wetted with water to generate protons, and many colorants are discolored by receiving protons.

Examples of the anionic surfactant include:
alkali metal alkyl sulfates such as sodium dodecyl sulfate and potassium dodecyl sulfate;
sodium dodecyl polyglycol ether sulfate;
ammonium alkyl sulfates such as ammonium dodecyl sulfate;
sodium sulfosinoate;
alkyl sulfonates such as alkali metal salts of sulfonated paraffin and ammonium salts of sulfonated paraffin;
fatty acid salts such as sodium laurate, triethanolamine oleate and triethanolamine abietate;
alkylaryl sulfonates such as sodium dodecylbenzene sulfonate and alkali metal sulfates of alkali phenol hydroxyethylene;
high alkyl naphthalene sulfonates;
naphthalenesulfonic acid formalin condensate;
dialkyl sulfosuccinates;
polyoxyethylene alkyl sulfate salts;
polyoxyethylene alkylaryl sulfate salts, and the like.

Examples of the nonionic surfactant include:
polyoxyethylene alkyl ethers;
polyoxyethylene alkylaryl ethers;
sorbitan fatty acid ester;
polyoxyethylene sorbitan fatty acid ester;
fatty acid monoglyceride such as glycerol monolaurate;
polyoxyethyleneoxypropylene copolymer;
condensation product of ethylene oxide and aliphatic amine, amide or acid, and the like.

The surfactant is preferably an anionic surfactant, more preferably a linear alkylbenzene sulfonate, and still more preferably sodium dodecylbenzene sulfonate. When sodium dodecylbenzene sulfonate is used as the surfactant, the colorant is easily dissolved in the oily substance, and the oily substance is so easily brought into contact with water, that protons are easily generated. Since protons are easily provided to the colorant, discoloration of the composition for wet indicator rapidly proceeds.

The surfactant is contained in the composition for wet indicator in an amount of preferably from 1 to 50% by mass, more preferably from 5 to 30% by mass, and still more preferably from 10 to 25% by mass, based on the total amount of the oily gel, the colorant and the water holding agent. By adjusting the content of the surfactant in the composition for wet indicator within the above ranges, sensitivity of the composition for wet indicator to moisture is improved, the degree of discoloration is made clear, and a wet state is more accurately displayed.

### <Pigment>

In the composition for wet indicator of the present invention, a pigment may be used as necessary. The pigment includes inorganic pigments and organic pigments. The inorganic pigment includes colored inorganic pigments and extender pigments.

Examples of the colored inorganic pigment include white titanium oxide, white lead (basic lead carbonate), zinc white (zinc oxide), lithopone (barium sulfate/zinc sulfide);
reddish colcothar (red iron (III) oxide), red lead (lead oxide), silver vermilion (mercury sulfide), molybdenum-red;
yellowish chrome yellow (lead chromate), cadmium yellow (cadmium sulfide), zinc chromate, litharge (lead monoxide);
bluish ultramarine blue, Prussian blue, cobalt blue (cobalt aluminate);
black pigment includes iron black (iron (II, III) oxide, carbon black and the like.

Examples of the extender pigment include barite (barium sulfate), gypsum (hydrous calcium sulfate), kaolin (white clay), silica (silicon dioxide), white carbon (precipitated silica), talc, barium carbonate, calcium carbonate and the like.

Examples of the organic pigment include lake and colored organic pigments. There are two types of lake, one of which is dyeing lake, which is a pigment obtained by dyeing an extender pigment with a dye. Another lake is one in which a dye is made insoluble by reacting with a divalent or higher metal salt, and examples thereof include azo lake formed from an azo dye.

Examples of the colored organic pigment include insoluble azo pigments, metal phthalocyanine pigments, anthraquinone pigments, vat pigments and the like. Among azo dyes, anthraquinone dyes and indigo dyes shown as representative types of dyes based on chemical structures, substances insoluble in water are used as pigments. Moreover, the metal phthalocyanine pigment has a color tone from blue to green, and is excellent in light resistance.

### <Tackifier Resin>

In the composition for wet indicator of the present invention, a tackifier resin is preferably used as necessary. By including the tackifier resin, the composition for wet indicator may maintain a balance between the odor reduction and the suppression of the elution of color.

Examples of the tackifier resin include copolymers of natural terpenes, three-dimensional polymers of natural terpenes, hydrogenated derivatives of copolymers of hydrogenated terpenes, polyterpene resins, hydrogenated derivatives of phenol-based modified terpene resins, aliphatic petroleum hydrocarbon resins, hydrogenated derivatives of aliphatic petroleum hydrocarbon resins, aromatic petroleum hydrocarbon resins, hydrogenated derivatives of aromatic petroleum hydrocarbon resins, cyclic aliphatic petroleum hydrocarbon resins and hydrogenated derivatives of cyclic aliphatic petroleum hydrocarbon resins.

As the tackifier resin, a commercially available product may be used. Examples of such a commercially available product include KE-604 (trade name), ARKON P100 (trade name) and ARKON M100 (trade name) manufactured by Arakawa Chemical Industries, Ltd., RHR-101HK manufactured by Wuzhou Sun Shine Forestry & Chemicals Co., Ltd.,

FTR6100 manufactured by Mitsui Chemicals, Inc., CLEARON M105 (trade name) manufactured by YASUHARA CHEMICAL CO., LTD., ECR5600 (trade name), ECR5400 (trade name) and ECR179EX (trade name) manufactured by Exxon Mobil Corporation, Quinton DX390 (trade name) manufactured by Zeon Corporation and the like. These commercially available tackifier resins may be used alone or in combination.

The tackifier resin is not particularly limited, but is contained in the composition for wet indicator in an amount of preferably 300% by mass or less, more preferably from 10 to 250% by mass, and still more preferably from 25 to 200% by mass, based on the total amount of the oily gel, the colorant, and the water holding agent.

### <Other Components>

The composition for wet indicator of the present invention may contain, as components other than the above, at least one additive of a thickener (styrene-based polymer, olefin-based polymer), an antioxidant (phenol-based, phosphorus-based and sulfur-based), an ultraviolet absorber, a fluorescent brightening agent, a non-discoloring dye, a fragrance, a disinfectant, an antibacterial agent, a repellent, a skin care component, a non-discoloring pigment, a lubricant and a filler (including microcapsules).

### <Method for Producing Composition for Wet Indicator>

The composition for wet indicator of the present invention is prepared by mixing the above-described components with heating as necessary. When the components are mixed until they become uniform and cooled to room temperature, the composition for wet indicator is converted into gel to become hard.

For example, all the components contained in the composition for wet indicator are put into a container, and the components are dissolved or uniformly dispersed, and heated and stirred until a uniform state is obtained. The heating temperature is appropriately adjusted generally in the range of from 50 to 250° C., preferably from 70 to 200° C., and more preferably from 80 to 160° C. The heating time is determined in consideration of the heating temperature, but is generally adjusted between 5 minutes and 1 hour, and preferably between 10 minutes and 40 minutes. A uniform composition may be prepared by putting each component into a container in turn and sequentially mixing the components.

### <Wet Indicator>

The composition for wet indicator is molded into a suitable shape and is used as a wet indicator, optionally in combination with a component, substance or material that does not impair wet indicator function. The composition for wet indicator may be used as a single material, for example, by being applied onto a substrate material such as a film or paper, or may be used in combination with a water-absorbent substance. The wet indicator of the present invention may be used for various articles that require detecting a wet state, but is particularly preferably used for absorbent articles.

When the composition for wet indicator and the water-absorbent substance are combined, examples of embodiment thereof are as follows. That is, positioning a composition for wet indicator and a water-absorbent substance adjacent to each other;
mixing the composition for wet indicator and the water-absorbent substance; and
heating the composition for wet indicator and the water-absorbent substance to be compatible with each other.

The water-absorbent substance may be a known resin having a water-absorbing property such as polyvinyl alcohol or an acrylic resin or a known substance having a water-absorbing property such as wood, paper or cloth. The shape of the water-absorbent substance may be any shape such as a sheet shape, a lump shape, a granular shape or a fibrous shape.

### <Absorbent Article>

The absorbent article of the present invention includes a water-absorbent substance and the composition for wet indicator of the present invention. Specifically, absorbent articles are so-called hygienic materials such as a sanitary napkin, a urine absorbing liner, puerperal shorts, a breast milk pad, an armpit sweat pad, a paper diaper, a pet sheet, a hospital gown and a surgical white gown.

The absorbent article is composed of at least one member selected from the group consisting of a woven fabric, a nonwoven fabric, a rubber, a resin, paper and a polyolefin film, and the wet indicator according to the present invention. The polyolefin film is preferably a polyethylene film for reasons such as durability and cost.

When the wet indicator of the present invention is used for a paper diaper, the wet indicator is preferably attached to the surface of the powder or particles of the water-absorbent resin stored in the paper diaper. In addition to the paper diaper, by applying the wet indicator of the present invention to surfaces of various substrate materials such as resin particles, woven fabric, nonwoven fabric, resin sheet, paper, resin molded article, metal and wood by any means, a moisture wet indicator action may be provided on the surface of various substrate materials.

### EXAMPLES

Hereinafter, for the purpose of describing the present invention in more detail and specifically, the present invention will be described using examples, but these examples do not limit the present invention at all. In Examples and Comparative Examples, components to be blended in a composition for wet indicator are shown below.

Gelling agents and oily substances constituting the oily gel are as follows.
(A) Gelling Agents (saturated fatty acids having 16 or more carbon atoms or derivatives thereof)
(A1) Saturated fatty acid having 18 carbon atoms: 12-Hydroxystearic acid (reagent name) manufactured by ITOH OIL CHEMICALS CO., LTD.
(A2) Glyceride of saturated fatty acids and unsaturated fatty acids: Castor hardened oil A (trade name) manufactured by ITOH OIL CHEMICALS CO., LTD.
(A3) Saturated fatty acid having 18 carbon atoms: Stearic acid (reagent name) manufactured by FUJIFILM Wako Pure Chemical Corporation)
(A′4) Unsaturated fatty acid having 18 carbon atoms: Oleic acid (reagent name) manufactured by FUJIFILM Wako Pure Chemical Corporation), which is a non-gelling agent having no function of converting liquid oily substance into gel.
(B) Liquid Oily Substances
(B1) Liquid paraffin: Pharmacopoeial liquid paraffin (reagent name) manufactured by FUJIFILM Wako Pure Chemical Corporation
(B2) Paraffin oil: Daphne Oil KP-68 (trade name) manufactured by Idemitsu Kosan Co., Ltd.
(B3) Naphthene oil: KNH4010 (trade name) manufactured by PetroChina Company Limited
(B′4) Non-oily substance: CITROFOL AO (trade name) manufactured by Jungbunzlauer AG, the non-oily substance is not compatible with oil, and does not have the properties as an oil.
(B′5) Non-oily substance: Benzoflex 9-88 (trade name) manufactured by Eastman Chemical Company, the non-oily substance is not compatible with oil, and does not have the properties as an oil.
(C) Colorants
(C1) Leuco dye: Crystal violet lactone (trade name) manufactured by FUJIFILM Wako Pure Chemical Corporation, phthalides
(C2) Leuco dye: Black 400 (trade name) manufactured by Fukui Yamada Chemical Co., Ltd., fluorans
(C3) Leuco dye: Red 50 (trade name) manufactured by Fukui Yamada Chemical Co., Ltd., fluorans
(C4) pH Indicator: Bromocresol green (trade name) manufactured by FUJIFILM Wako Pure Chemical Corporation
(D) Water Holding Agents
(D1) Higher alcohol derivative having 18 carbon atoms (stearyl alcohol): KALCOL 8098 (trade name) manufactured by Kao Corporation
(D2) Higher alcohol derivative having 18 carbon atoms (stearyl alcohol): 1-Octadecanol (reagent name) manufactured by FUJIFILM Wako Pure Chemical Corporation
(D3) Higher alcohol derivative having 16 carbon atoms (cetyl alcohol): 1-Hexadecanol (reagent name) manufactured by FUJIFILM Wako Pure Chemical Corporation
(D4) Higher alcohol (1-hexacosanol) derivative having 26 carbon atoms: LICOCARE RBW 106 TP (trade name) manufactured by Clarient (Japan) K. K., rice brown wax
(D5) Higher alcohol (1-hexacosanol) derivative having 26 carbon atoms: LICOCARE RBW 102 TP (trade name) manufactured by Clarient (Japan) K. K., rice brown wax
(D6) Higher alcohol derivative having from 26 to 30 carbon atoms: Carnauba wax No. 1 (trade name) manufactured by Yamakei Co., Ltd., carnauba wax
(D′7) Alkanes: Octadecane (reagent name) manufactured by Fujifilm Wako Pure Chemicals, which is a non-water holding agent having no function of holding water.
(D′8) Fischer-Tropsch wax: Sasol C80 (trade name) manufactured by Sasol Corporation, non-water holding agent
(E) Surfactants
(E1) Anionic surfactant: Sodium dodecylbenzene sulfonate (reagent name) manufactured by KANTO CHEMICAL CO., INC.
(E2) Anionic surfactant: Sodium alkylbenzene sulfonate, LAS-P95 (trade name) manufactured by Jiangsu Qingting Washing Products Co., Ltd.
(E3) Anionic surfactant: Sodium alkyldiphenyl ether sulfonate powder Perex SS-L (trade name) manufactured by Kao Corporation
(E4) Nonionic surfactant: Castor oil fatty acid polyethylene glycol ester AQ-250 (trade name) manufactured by ITO OIL CHEMICALS CO., LTD.
(F) Tackifier Resins
(F1) Hydrogenated petroleum resin: ARKON M100 (trade name) manufactured by Arakawa Chemical Industries, Ltd.
(F2) Hydrogenated petroleum resin: HA-100 (trade name) manufactured by Henghe Material Technology Co., Ltd.
(F3) Hydrogenated petroleum resin: T-REZ HA103 (trade name) manufactured by ENOS Corporation
(F4) Hydrogenated rosin ester: Foral 85E (trade name) manufactured by Eastman Chemical Company
(F5) Aliphatic hydrocarbon resin: Quinton CX495 (trade name) manufactured by Zeon Corporation

The above-described components were blended in ratios shown in Tables 1 to 5, and stirred and mixed to prepare a composition for wet indicator. Specifically, components were placed in a 70 ml container and heated to 130° C. with a glass-col heater, and a blend of the components was stirred using a stirrer at a stirring speed of from 300 to 500 rpm for 20 minutes. All numerical values relating to the composition for wet indicator (composition) shown in Tables 1 to 5 are parts by mass (solid content).

TABLE 1

| | | Examples | | | | | |
|---|---|---|---|---|---|---|---|
| Blending components | | 1 | 2 | 3 | 4 | 5 | 6 |
| Gelling | 12-Hydroxystearic acid | | | 24 | 5 | | 30 |
| agent | Castor hardened oil (castor | | 24 | | | 20 | |
| | hardened oil A) | | | | | | |
| | Stearic acid | 24 | | | | | |
| Liquid | Paraffin oil (liquid paraffin) | 65 | | | 84 | | |
| oily | Paraffin oil (KP-68) | | 65 | | | 69.5 | |
| substance | Naphthene oil (KN4010) | | | 65 | | | 59.8 |
| Colorant | Leuco dye (crystal violet | | | 1 | 1 | | |
| | lactone) | | | | | | |
| | Leuco dye (Black400) | | | | | 0.5 | |
| | Leuco dye (Red500) | | 1 | | | | 0.2 |
| | pH Indicator (bromocresol | 1 | | | | | |
| | green) | | | | | | |
| Water | Stearyl alcohol (KALCOL | | | | 10 | | |
| holding | 8098) | | | | | | |
| agent | Stearyl alcohol (1- | | | | | | |
| (higher | octadecanol) | | | | | | |
| alcohol | Cetyl alcohol (1- | 10 | | | | | |
| derivative) | hexadecanol) | | | | | | |
| | Hexacosanol derivative | | | | | 10 | |
| | (LICOCARE RBM 106 TP) | | | | | | |
| | Hexacosanol derivative | | 10 | | | | 10 |
| | (LICOCARE RBM 102 TP) | | | | | | |
| | Carnauba wax (carnauba | | | 10 | | | 0 |
| | wax No. 1) | | | | | | |
| Surfactant | Sodium dodecylbenzene | | | 10 | 10 | 15 | 10 |
| | sulfonate | | | | | | |
| | Sodium alkylbenzene | | 10 | | | | |
| | sulfonate | | | | | | |
| | Sodium alkyldiphenyl ether | | | | | | |
| | disulfonate | | | | | | |
| | Castor oil fatty acid PEG | 10 | | | | | |
| | ester (AQ-250) | | | | | | |
| Tackifier | Hydrogenated petroleum | | | | 175 | | |
| resin | resin (ARKON M100) | | | | | | |
| | Hydrogenated petroleum | | | | | 150 | |
| | resin (HA-100) | | | | | | |
| | Hydrogenated petroleum | | | | | | 100 |
| | resin (T-REZ HA103) | | | | | | |
| Total | Oily gel component + | 100 | 100 | 100 | 100 | 100 | 100 |
| | Colorant + Water holding | | | | | | |
| | agent | | | | | | |
| | Oily gel component | 90 | 90 | 90 | 90 | 90 | 90 |
| | All blended | 110 | 110 | 110 | 285 | 265 | 210 |

TABLE 2

| | | Examples | | | | | |
|---|---|---|---|---|---|---|---|
| Blending components | | 7 | 8 | 9 | 10 | 11 | 12 |
| Gelling | 12-Hydroxystearic acid | 70 | 20 | | 20 | 20 | 46 |
| agent | Castor hardened oil (castor | | | 20 | | | |
| | hardened oil A) | | | | | | |
| | Stearic acid | | | | | | |
| Liquid | Paraffin oil (liquid paraffin) | 17 | | | | | |
| oily | Paraffin oil (KP-68) | | | 74.9 | 53 | 69 | 42 |
| substance | Naphthene oil (KN4010) | | 78 | | | | |
| Colorant | Leuco dye (crystal violet | | 1 | | 7 | | 1 |
| | lactone) | | | | | | |
| | Leuco dye (Black400) | 3 | | 0.1 | | | |
| | Leuco dye (Red500) | | | | | 1 | |
| | pH Indicator (bromocresol | | | | | | |
| | green) | | | | | | |
| Water | Stearyl alcohol (KALCOL | 10 | 1 | | | 10 | 11 |
| holding | 8098) | | | | | | |
| agent | Stearyl alcohol (1- | | | | | | |
| (higher | octadecanol) | | | | | | |
| alcohol | Cetyl alcohol (1- | | | | | | |
| derivative) | hexadecanol) | | | | | | |
| | Hexacosanol derivative | | | 5 | | | |
| | (LICOCARE RBM 106 TP) | | | | | | |
| | Hexacosanol derivative | | | | 20 | | |
| | (LICOCARE RBM 102 TP) | | | | | | |
| | Carnauba wax (carnauba | | | | | | |
| | wax No. 1) | | | | | | |
| Surfactant | Sodium dodecylbenzene | | 10 | 20 | 5 | 10 | |
| | sulfonate | | | | | | |
| | Sodium alkylbenzene | | | | | | 14 |
| | sulfonate | | | | | | |
| | Sodium alkyldiphenyl ether | | | | | | |
| | disulfonate | | | | | | |
| | Castor oil fatty acid PEG | | | | | | |
| | ester (AQ-250) | | | | | | |
| Tackifier | Hydrogenated petroleum | | | 200 | | 125 | |
| resin | resin (ARKON M100) | | | | | | |
| | Hydrogenated petroleum | 25 | | | 100 | | 115 |
| | resin (HA-100) | | | | | | |
| | Hydrogenated petroleum | | 10 | | | | |
| | resin (T-REZ HA103) | | | | | | |
| Total | Oily gel component + | 100 | 100 | 100 | 100 | 100 | 100 |
| | Colorant + Water holding | | | | | | |
| | agent | | | | | | |
| | Oily gel component | 87 | 98 | 95 | 73 | 89 | 88 |
| | All blended | 125 | 120 | 320 | 205 | 235 | 229 |

TABLE 3

| | | Examples | | | |
|---|---|---|---|---|---|
| Blending components | | 13 | 14 | 15 | 16 |
| Gelling | 12-Hydroxystearic acid | 32 | 48 | 43 | 23 |
| agent | Castor hardened oil | | | | |
| | (castor hardened oil A) | | | | |
| | Stearic acid | | | | |
| Liquid | Paraffin oil (liquid paraffin) | | | | |
| oily | Paraffin oil (KP-68) | 27 | 44 | 32 | 52 |
| substance | Naphthene oil (KN4010) | | | | |
| Colorant | Leuco dye | 1 | 1 | 1 | 2 |
| | (crystal violet lactone) | | | | |
| | Leuco dye (Black400) | | | | |
| | Leuco dye (Red500) | | | | |
| | pH Indicator | | | | |
| | (bromocresol green) | | | | |
| Water | Stearyl alcohol | | | | 23 |
| holding | (KALCOL 8098) | | | | |
| agent | Stearyl alcohol | | | | |
| (higher | (1-octadecanol) | | | | |
| alcohol | Cetyl alcohol | | | | |
| derivative) | (1-hexadecanol) | | | | |
| | Hexacosanol derivative | 40 | 7 | 24 | |
| | (LICOCARE RBM 106 TP) | | | | |
| | Hexacosanol derivative | | | | |
| | (LICOCARE RBM 102 TP) | | | | |
| | Carnauba wax (carnauba | | | | |
| | wax No. 1) | | | | |
| Surfactant | Sodium dodecylbenzene | | | | |
| | sulfonate | | | | |
| | Sodium alkylbenzene | 13 | 19 | | |
| | sulfonate | | | | |
| | Sodium alkyldiphenyl ether | | | 26 | 50 |
| | disulfonate | | | | |
| | Castor oil fatty acid PEG | | | | |
| | ester (AQ-250) | | | | |
| Tackifier | Hydrogenated petroleum | 48 | 120 | | |
| resin | resin (ARKON M100) | | | | |
| | Hydrogenated petroleum | | | 102 | 125 |
| | resin (HA-100) | | | | |
| | Hydrogenated petroleum | | | | |
| | resin (T-REZ HA103) | | | | |
| Total | Oily gel component + | 100 | 100 | 100 | 100 |
| | Colorant + Water holding | | | | |
| | agent | | | | |
| | Oily gel component | 59 | 92 | 75 | 75 |
| | All blended | 161 | 239 | 228 | 275 |

TABLE 4

| | | Comparative Examples | | | | | |
|---|---|---|---|---|---|---|---|
| Blending components | | 1 | 2 | 3 | 4 | 5 | 6 |
| Gelling | 12-Hydroxystearic acid | | | 24 | 24 | 24 | 24 |
| agent | Oleic acid (non-gelling | | 24 | | | | |
| | agent) | | | | | | |
| Liquid | Paraffin oil (liquid paraffin) | 89 | 65 | | 75 | 65 | 65 |
| oily | Non-oily substance | | | 65 | | | |
| substance | (CITROFOL AO) | | | | | | |
| | Non-oily substance | | | | | | |
| | (Benzoflex 9-98) | | | | | | |
| Colorant | Leuco dye (crystal violet | 1 | 1 | 1 | 1 | 1 | 1 |
| | lactone) | | | | | | |
| Water | Stearyl alcohol (KALCOL | 10 | 10 | 10 | | | |
| holding | 8098) | | | | | | |
| agent | | | | | | | |
| (higher | Octadecane (non-water | | | | | 10 | |
| alcohol | holding agent) | | | | | | |
| derivative) | Fischer-Tropsch wax | | | | | | 10 |
| | (Sasol C80, non-water | | | | | | |
| | holding agent) | | | | | | |
| Surfactant | Sodium dodecylbenzene | 10 | 10 | 10 | 10 | 10 | 10 |
| | sulfonate | | | | | | |
| | Hydrogenated petroleum | | | | | | |
| | resin (ARKON M100) | | | | | | |
| Tackifier | Hydrogenated rosin ester | | | | | | |
| resin | (Foral 85E) | | | | | | |
| | Aliphatic hydrocarbon | | | | | | |
| | resin (Quinton CX495) | | | | | | |
| Total | Oily gel component + | 100 | 100 | 100 | 100 | 100 | 100 |
| | Colorant + Water holding | | | | | | |
| | agent | | | | | | |
| | All blended | 110 | 110 | 110 | 110 | 110 | 110 |

TABLE 5

| | | Comparative Examples | | |
|---|---|---|---|---|
| Blending components | | 7 | 8 | 9 |
| Gelling | 12-Hydroxystearic acid | 24 | | 24 |
| agent | Oleic acid | | | |
| | (non-gelling agent) | | | |
| Liquid | Paraffin oil (liquid paraffin) | 65 | 89 | 75 |
| oily | Non-oily substance | | | |
| substance | (CITROFOL AO) | | | |
| | Non-oily substance | | | |
| | (Benzoflex 9-88) | | | |
| Colorant | Leuco dye | 1 | 1 | 1 |
| | crystal violet lactone) | | | |
| Water | (Stearyl alcohol | | 10 | |
| holding | (KALCOL 8098) | | | |
| agent | Octadecane (non-water | | | |
| (higher | holding agent) | | | |
| alcohol | Fischer-Tropsch wax | | | |
| derivative) | (Sasol C80, non-water | | | |
| | holding agent) | | | |
| Surfactant | Sodium dodecylbenzene | 10 | 10 | 10 |
| | sulfonate | | | |
| | Hydrogenated petroleum | | 100 | |
| | resin (ARKON M100) | | | |
| Tackifier | Hydrogenated rosin ester | 10 | | |
| resin | (Foral 85E) | | | |
| | Aliphatic hydrocarbon | | | 100 |
| | resin (Quinton CX495) | | | |
| Total | Oily gel component + | 90 | 100 | 100 |
| | Colorant + Water holding | | | |
| | agent | | | |
| | All blended | 110 | 210 | 210 |

For the compositions for wet indicator of Examples and Comparative Examples, appearance was confirmed whether or not an oily gel was formed, and further, phase separation, odor, color phase change, discoloration time and color elution were evaluated. Details of the evaluation are shown below. The evaluation results are as shown in Tables 6 to 10.

### <Appearance Properties>

The compositions for wet indicator of Examples and Comparative Examples were allowed to stand at room temperature for 1 day, and appearance of each composition was visually observed to confirm whether or not an oily gel was generated. Whether or not the composition for wet indicator was a gel was evaluated by fluidity of the composition and oozing of the oily substance.

The composition was placed in a container, the composition was not flowable even when the container was tilted, and a commercially available oil blotting paper was pressed against the composition, and when oil oozing was visually recognized, the composition was determined as a gel.

Hardness of the composition for wet indicator was evaluated according to the following criteria. In an environment of 23° C., a cylindrical SUS probe was pressed against the composition for wet indicator of each of Examples and Comparative Examples to apply a load of 1 kg, and after 8 seconds, the degree of penetration of the SUS probe into the composition was confirmed to evaluate the hardness of the composition. The evaluation criteria are as follows.

Penetration of probe having a diameter of 16 mm is 4 mm or more: Very soft Penetration of probe having a diameter of 16 mm is less than 4 mm: Soft Penetration of the probe having a diameter of 5 mm is less than 4 mm: Hard Penetration of probe having a diameter of 3 mm is less than 4 mm: Very hard

### <Phase Separation>

Using 30 g of the composition for wet indicator of each of Examples and Comparative Examples as an evaluation sample, 30 g of each sample was placed in a 70 ml glass container and allowed to stand in a heating furnace at 100° C. After aging the sample for 24 hours, the presence or absence of phase separation was visually confirmed to evaluate whether the composition was uniform or non-uniform.
A (Excellent): No phase separation is observed, and no turbidity is observed
B (Good): Phase separation is not observed and is uniform, but slight turbidity is observed
C (Fair): A slightly transparent supernatant is observed at the top
D (Fail): Obvious phase separation is observed

### <Odor Evaluation>

### ·Odor at Normal Temperature (23° C.)

Using 30 g of the composition for wet indicator of each of Examples and Comparative Examples as an evaluation sample, 30 g of each sample was placed in a 70 ml glass container, covered with an aluminum foil, and then allowed to stand for 24 hours in a thermostatic chamber at 23° C. Thereafter, the container was taken out from a dryer, the aluminum foil was removed, and odor was confirmed. The evaluation criteria are as follows.
A (Excellent): No odor is sensed
B (Good): A slight odor is sensed
C (Fair): An obvious odor is sensed but is not unpleasant
D (Fail): A strong and unpleasant odor is sensed

### ·Odor on Heating (120° C.)

Using 30 g of the composition for wet indicator of each of Examples and Comparative Examples as an evaluation sample, 30 g of each sample was placed in a 70 ml glass container, covered with an aluminum foil, and then allowed to stand for 1 hour in a dryer at 120° C. Thereafter, the container was taken out from the dryer, the aluminum foil was removed, and odor was confirmed. The evaluation criteria are as follows.
A (Excellent): No unpleasant odor is sensed
B (Good): A slight unpleasant odor is sensed
C (Fair): An unpleasant odor is sensed
D (Fail): A strong and unpleasant odor is sensed

### <Initial Hue>

The wet indicator composition of each of Examples and Comparative Examples was placed in a glass bottle, and the glass bottle was heated in a dryer at 100° C. until a uniform liquid was obtained. Subsequently, an appropriate amount of each composition was dropped on a hiding rate test paper (white reflectance: 80±2%, manufactured by Hanchen Corporation), then a uniform film having a thickness of about 27.5 μm was quickly prepared with a bar coater (No. 12 manufactured by Dai-ichi Rika Co., Ltd.), and color of this coated film was recorded as an initial state (sample).

<Hue after Water Wetting>

The sample was allowed to stand at room temperature for 30 minutes or more and then placed on a table, and distilled water was sprayed on the sample. Thereafter, final color, the degree of hue change (color difference) and time required for changing to the final color observed by visual observation were recorded.

### <Color Difference (Degree of Hue Change)>

Evaluation criteria of the hue change are as follows.
A (Excellent): A very clear difference in hue is observed before and after spraying
B (Good): A clear difference in hue is observed before and after spraying
C (Fair): A slight difference in hue is observed before and after spraying
D (Fail): No difference in hue is observed before and after spraying

### <Time Until Discoloration>

The time until becoming the final color after water was sprayed was measured. It is to be noted that, for compositions in which no color difference was observed, symbol “-” was shown in the table.

### <Color Elution>

Five grams of the composition for indicator was heated and melted at 100° C., poured into a 7 cm diameter foamed polystyrene container, and after 1 hour, 1 mL of saline solution (0.9% salt concentration) was added to the composition for indicator in the container and allowed to stand for 30 seconds.

The added saline solution was transferred to another foamed polystyrene container, and the degree of coloration was confirmed by visual observation. If coloration was observed, the saline solution was transferred onto white drawing paper (thick drawing paper manufactured by Kyowa Shiko Co., Ltd.), and the saline solution was spread over 20 cm long, 20 cm wide, and approximately 25 μm thick to confirm the degree of coloration. The evaluation criteria are as follows.
A (Excellent): No coloration is observed in the saline solution in the polystyrene container
B (Good): Slight coloration is observed in the saline solution in the polystyrene container, but no coloration is observed on the drawing paper
C (Fair): Slight coloration is observed in the saline solution in the polystyrene container, and slight coloration is also observed on the drawing paper
D (Fail): Obvious coloration is observed also on the drawing paper

TABLE 6

| | | Examples | | | | | |
|---|---|---|---|---|---|---|---|
| Blending components | | 1 | 2 | 3 | 4 | 5 | 6 |
| Performance | Appearance properties | Soft gel | Hard gel | Hard gel | Soft gel | Hard gel | Hard gel |
| evaluation | Phase separation | A | A | A | A | A | A |
| results | (100° C. for one day) | | | | | | |
| | Odor at room | C | A | A | A | A | A |
| | temperature (23° C.) | | | | | | |
| | Odor on heating (120° C.) | C | A | A | A | B | A |
| | Initial hue | Yellow | Impercep- | Impercep- | Impercep- | Impercep- | Impercep- |
| | | | tible | tible | tible | tible | tible |
| | Hue after water wetting | Green | Red | Blue | Thin blue | Black | Red |
| | Color difference | B | A | A | C | A | A |
| | Time until discoloration | Immediate | Immediate | Immediate | 10 | 10 | Immediate |
| | | | | | Seconds | Seconds | |
| | Color elution | A | C | C | C | A | C |

TABLE 7

| | | Examples | | | | | |
|---|---|---|---|---|---|---|---|
| Blending components | | 7 | 8 | 9 | 10 | 11 | 12 |
| Performance | Appearance properties | Hard gel | Soft gel | Hard gel | Hard gel | Hard gel | Hard gel |
| valuation | Phase separation | A | A | A | A | A | A |
| results | (100° C. for one day) | | | | | | |
| | Odor at room | A | A | A | A | A | A |
| | temperature (23° C.) | | | | | | |
| | Odor on heating | B | A | A | B | A | A |
| | (120° C.) | | | | | | |
| | Initial hue | Thin | Impecep- | Impercep- | Thin blue | Impercep- | Impercep- |
| | | black | tible | tible | | tible | tible |
| | Hue after water wetting | Black | Blue | Black | Blue | Red | Blue |
| | Color difference | C | A | C | B | A | A |
| | Time until discoloration | Immediate | Immediate | Immediate | Immediate | Immediate | Immediate |
| | Color elution | A | C | A | A | A | A |

TABLE 8

| | | Examples | | | |
|---|---|---|---|---|---|
| Blending components | | 13 | 14 | 15 | 16 |
| Performance | Appearance | Hard | Hard | Hard | Hard |
| valuation | properties | gel | gel | gel | gel |
| results | Phase separation | A | A | A | A |
| | (100° C. for | | | | |
| | one day) | | | | |
| | Odor at room | A | A | A | A |
| | temperature | | | | |
| | (23° C.) | | | | |
| | Odoron heating | A | A | A | A |
| | (120° C.) | | | | |
| | Initial hue | Imper- | Imper- | Imper- | Imper- |
| | | ceptible | ceptible | ceptible | ceptible |
| | Hue after water | Blue | Blue | Blue | Blue |
| | wetting | | | | |
| | Color difference | A | A | A | A |
| | Time until | Im- | Im- | Im- | Im- |
| | discoloration | mediate | mediate | mediate | mediate |
| | Color elution | A | A | A | A |

TABLE 9

| | | Comparative Examples | | | | | |
|---|---|---|---|---|---|---|---|
| Blending components | | 1 | 2 | 3 | 4 | 5 | 6 |
| Performance | Appearance | Non- | Non- | Non- | Hard gel | Non- | Non- |
| evaluation | | uniform | uniform | uniform | | uniform | uniform |
| results | | liquid | liquid | solid | | solid | solid |
| | Phase separation | D | A | D | D | D | D |
| | (100° C. for one | | | | | | |
| | day) | | | | | | |
| | Odor at room | A | A | C | A | A | A |
| | temperature | | | | | | |
| | (23° C.) | | | | | | |
| | Odor on heating | A | D | C | B | D | A |
| | (120° C.) | | | | | | |
| | Initial hue | Impercep- | Impercep- | Impercep- | Impercep- | Impercep- | Impercep- |
| | | tible | tible | tible | tible | tible | tible |
| | Hue after water | Blue | Thin blue | Impercep- | Blue | Blue | Impercep- |
| | wetting | | | tible | | | tible |
| | Color difference | A | B | D | A | A | D |
| | Time until | Immediate | Immediate | — | Immediate | 30 | — |
| | discoloration | | | | | Seconds | |
| | | | | | | or more | |
| | Color elution | D | D | D | A | A | A |

TABLE 10

| | | Comparative Examples | | |
|---|---|---|---|---|
| Blending components | | 7 | 8 | 9 |
| Performance | Appearance | Non- | Viscous | Soft gel |
| evaluation | | uniform | liquid | |
| results | | gel | | |
| | Phase separation | D | D | D |
| | (100° C. for one day) | | | |
| | Odor at room | D | A | D |
| | temperature (23° C.) | | | |
| | Odor on heating | D | A | D |
| | (120° C.) | | | |
| | Initial hue | Imper- | Imper- | Imper- |
| | | ceptible | ceptible | ceptible |
| | Hue after water | Imper- | Thin | Blue |
| | wetting | ceptible | blue | |
| | Color difference | D | C | B |
| | Time until | — | 30 | Immediate |
| | discoloration | | Seconds | |
| | | | or more | |
| | Color elution | D | A | A |

As shown in Tables 6 to 8, the compositions for wet indicator of the examples have no D in the evaluation items. There are C's in the evaluation of some examples, but overall, the compositions for wet indicator of the examples have gel-like appearance, the respective components do not cause phase separation, the odor can be reduced, the color difference due to water wetting is clear, the time until discoloration is short, and also the color elution is low.

In contrast, the compositions for wet indicator of the comparative examples have a D in any one of the evaluation items, as shown in Tables 9 and 10.

Thus, it was confirmed that the composition for wet indicator having all the components of the hydrophilic gel, colorant, and water retention agent has an excellent balance of the above-mentioned performance and can be suitably used for absorbent articles.

### INDUSTRIAL APPLICABILITY

The composition for wet indicator of the present invention provides a wet indicator to be attached to an absorbent article such as a diaper or a napkin.

1. A composition for wet indicator comprising:
(A) an oily gel, which is a swollen body of a three-dimensionally crosslinked liquid oily substance and a gelling agent,
(B) a colorant and
(C) a water holding agent.

2. The composition for wet indicator according to claim 1, wherein the water holding agent is an aliphatic alcohol having 6 or greater carbon atoms or an aliphatic alcohol derivative having 6 or greater carbon atoms.

3. The composition for wet indicator according to claim 2, wherein the aliphatic alcohol derivative is selected from the group consisting of esters of stearyl alcohol, rice wax and carnauba wax.

4. A wet indicator comprising the composition for wet indicator according to claim 1.

5. An absorbent article comprising the wet indicator according to claim 4.

6. A composition for wet indicator comprising:
(A) 17-85% by mass of the liquid oily substance and a 10 to 50% by mass of the gelling agent,
(B) 0.1-4% by mass of the colorant, and
(C) 10-35% by mass of the water holding agent.

7. The composition for wet indicator according to claim 1, wherein the liquid oily substance is a liquid polymer selected from the group consisting of polybutene, polybutadiene, polyisobutylene and polyisoprene, and wherein the liquid polymer has a weight average molecular weight of from 2000 to 100000.

8. The composition for wet indicator according to claim 1, wherein the liquid polymer has a weight average molecular weight of from 2000 to 80000.

9. The composition for wet indicator according to claim 1, wherein the gelling agent is a saturated fatty acid having 16 or greater carbon atoms.

10. The composition for wet indicator according to claim 9, wherein the gelling agent is a saturated fatty acid having 16 to 36 carbon atoms.

11. The composition for wet indicator according to claim 10, wherein the gelling agent is selected from the group consisting of stearic acid having, 12-hydroxystearic acid, margaric acid, palmitic acid, and 16-hydroxyhexadecanoic acid.

12. The composition for wet indicator according to claim 1, wherein the colorant is a selected from the group consisting of oxazolidine-based dyes, azo-based dyes, methine-based dyes, anthraquinone-based dyes, leuco dyes, pH indicator, and mixtures thereof.

13. The composition for wet indicator according to claim 12, wherein the colorant is a mixture of leuco dye and pH indicator.

14. The composition for wet indicator according to claim 2, wherein the aliphatic alcohol or the aliphatic alcohol derivative has 16-34 carbon atoms.

15. The composition for wet indicator according to claim 2, wherein the aliphatic alcohol is selected from the group consisting of capryl alcohol (1-octanol), capric alcohol (1-decanol), lauryl alcohol, myristyl alcohol, tridecyl alcohol, cetanol, stearyl alcohol, ceryl alcohol (1-hexacosanol), myricyl alcohol (1-triacontanol), and gedyl alcohol (1-tetraacontanol).

16. The composition for wet indicator comprising according to claim 1, further comprising a surfactant, pigment, tackifier, thickener, antioxidant, ultraviolet absorber, florescent brightening agent, non-discolouring dye, fragrance, disinfectant, antibacterial agent, repellent, skin care component, non-discolouring pigment, lubricant or filler.

17. The composition for wet indicator comprising according to claim 16, wherein the surfactant is a linear alklbenzene sulfonate,
